(19) United States
(12) Patent Application Publication
(10) Pub. No.: US 2023/0416794 A1
(43) Pub. Date: Dec. 28, 2023

(54) Engineered Microorganisms for Producing Substituted Tryptamines

(71) Applicant: ALGAE-C INC., Caledon (CA)

(72) Inventors: Fatima Awwad, Trois-Rivieres (CA); William Mather Almon Carscallen, Halifax (CA); Elisa Ines Fantino, Trois-Rivieres (CA)

(21) Appl. No.: 18/036,768

(22) PCT Filed: Nov. 15, 2021
(86) PCT No.: PCT/CA2021/051617

§ 371 Date: May 12, 2023

Related U.S. Application Data
(60) Provisional application No. 63/114,145, filed on Nov. 16, 2020.

(51) Int. Cl. C12P 17/10; C12N 1/12; C12N 9/02; C12N 9/12; C12N 9/10; C12N 15/79; C12N 15/52

(52) U.S. Cl. CPC C12P 17/10; C12N 1/12; C12N 9/0083; C12N 9/1205; C12N 9/1007; C12N 9/1085; C12N 15/79; C12N 15/52; C12Y 201/01049; C12Y 114/16004; C12Y 205/01034; C12R 2001/89

(57) ABSTRACT

Provided herein are microorganisms for producing substituted tryptamines and cell cultures thereof, the microorganisms comprising at least one exogenous nucleic acid molecule that encodes at least one substituted tryptamine biosynthetic pathway enzyme.

### CROSS-REFERENCE TO RELATED APPLICATION

This Application claims the benefit of and priority from U.S. Provisional Patent Application No. 63/114,145, filed Nov. 16, 2020.

### FIELD

The present disclosure relates to microorganisms comprising an overexpressed tryptophan synthase, microorganisms comprising at least one nucleic acid molecule that encodes at least one substituted tryptamine biosynthetic pathway enzyme, and to substituted tryptamine biosynthetic pathway enzymes.

### BACKGROUND

Heterocyclic compounds have been proven to have important biological activity, especially indole derivatives. Important indole derivatives include substituted tryptamines that comprise a group of psychoactive compounds with properties of hallucinogens, neurotransmitters, and/or neuromodulators. The psychoactive properties of substituted tryptamines make them promising candidates for medicinal use, such as the treatment of depression, anxiety, or mental illness.

Substituted tryptamines such as psilocybin are produced naturally by fungi including members of the genus Psilocybe (e.g. P. cubensis and P. cyanescens). Culture of these fungi can be difficult and limiting to scaled production of psilocybin.

The production of psilocybin and other useful substituted tryptamines may be accelerated and rendered more economical by producing them in microorganisms that are easily culturable and that are capable of producing substituted tryptamines at larger scales.

### SUMMARY

The present disclosure provides a microorganism comprising an overexpressed tryptophan synthase.

The present disclosure further provides a microorganism comprising at least one nucleic acid molecule that encodes at least one substituted tryptamine biosynthetic pathway enzyme.

A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, an indole-ethylamine methyltransferase, a tryptamine 4-monooxygenase, and a 4-hydroxytryptamine kinase.

A microorganism comprising at least one exogenous nucleic acid molecule that encodes at least one substituted tryptamine biosynthetic pathway enzyme, wherein the at least one substituted tryptamine biosynthetic pathway enzyme comprises an indole-ethylamine methyltransferase, a tryptamine 4-monooxygenase, and a 4-hydroxytryptamine kinase.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a dimethylallyl tryptamine synthase.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, a tryptamine 5-hydroxylase, and an indole-ethylamine methyltransferase.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a tryptamine 5-hydroxylase, and an indole-ethylamine methyltransferase.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.

The present disclosure further provides a microorganism comprising at least one exogenous nucleic acid molecule that encodes at least one substituted tryptamine biosynthetic pathway enzyme, wherein the at least one substituted tryptamine biosynthetic pathway enzyme comprises a tryptophan halogenase and a tryptophan decarboxylase. The present disclosure further provides a substituted tryptamine biosynthetic pathway enzyme comprising an amino acid sequence with at least 80%, at least 85%, at least 90%, at least 95%, at least 99%, or 100% identity to the sequence as shown in any of SEQ ID NOs: 1-18 and 23-321.

The present disclosure further provides a tryptophan synthase comprising an amino acid sequence with at least 80%, at least 85%, at least 90%, at least 95%, at least 99%, or 100% identity to the sequence as shown in any of SEQ ID NOs: 19-22.

The present disclosure further provides a nucleic acid molecule encoding an enzyme as described herein.

The present disclosure further provides a vector comprising a nucleic acid molecule as described herein.

The present disclosure further provides a cell comprising a nucleic acid molecule or a vector as described herein.

The present disclosure further provides a cell culture comprising (i) the microorganism as described herein and (ii) a culture media optionally supplemented with a high concentration of tryptamine.

The present disclosure further provides a cell culture comprising (i) a microalga or a stramenopile comprising at least one exogenous nucleic acid molecule that encodes at least one substituted tryptamine biosynthetic pathway enzyme and (ii) a culture media supplemented with a high concentration of tryptamine.

The present disclosure further provides a method for producing at least one substituted tryptamine in a microalga or a stramenopile, comprising culturing the microalga or stramenopile in a culture media supplemented with a high concentration of tryptamine, wherein the microalga or stramenopile comprises at least one exogenous nucleic acid molecule that encodes at least one substituted tryptamine biosynthetic pathway enzyme.

### BRIEF DESCRIPTION OF THE DRAWINGS

The accompanying figures illustrate embodiments of the invention by way of example.

FIG. 1. Shows tryptamine (A) denoted with commonly substituted ‘R’ groups (B).

FIG. 2. Shows an exemplary substituted tryptamine biosynthetic pathway resulting in the production of psilocybin from L-tryptophan.

FIG. 3. Shows an exemplary substituted tryptamine biosynthetic pathway resulting in the production of serotonin from L-tryptophan (left), the production of psilocybin from L-tryptophan (center), and the production of DMT from L-tryptophan (right).

FIG. 4. Shows exemplary substituted tryptamine biosynthetic pathways for the production of psilocybin from L-tryptophan.

FIG. 5. Shows an exemplary substituted tryptamine biosynthetic pathway for the production of aurantioclavine from L-tryptophan.

FIG. 6. Shows an exemplary substituted tryptamine biosynthetic pathway for the production of bufotenine from L-tryptophan.

FIG. 7. Shows the survival rates as a percentage of live cells of different microorganism cultures (C. reinhardtii, P. tricomutum, S. limacinum, E. coli, and S. cerevisiae) supplemented with 2 mM of tryptamine for 36 hours after the addition of tryptamine to the culture media.

FIG. 8. Shows the survival rates as a percentage of live cells of different microorganism cultures (C. reinhardtii, P. tricomutum, S. limacinum, E. coli, and S. cerevisiae) supplemented with 5 mM of tryptamine for 36 hours after the addition of tryptamine to the culture media.

FIG. 9. Shows the relative growth of different microorganism cultures supplemented with 0.1 mM, 1 mM, or 10 mM of tryptamine measured at 16 hours (E. coli and S. cerevisiae) or 36 hours (C. reinhardtii, P. tricornutum, and S. limacinum) after the addition of tryptamine to the culture media. Relative growth calculated by comparing the cell density of each condition to the cell density of a corresponding control culture without supplemented tryptamine.

### DETAILED DESCRIPTION

### Tryptophan, Tryptamine, and Substituted Tryptamines

Tryptophan is a non-polar aromatic amino acid comprising an a-amino group, an α-carboxylic acid group, and a side chain indole. L-tryptophan is the L-isomer of tryptophan normally found in organisms. Tryptophan is derived from metabolites produced via glycolysis, the pentose phosphate pathway, and the shikimate pathway. Tryptophan synthase catalyzes the final steps of tryptophan synthesis. Tryptophan synthase consists of alpha and beta subunits. The alpha subunit catalyzes the formation of indole. The alpha subunit is responsible for the aldol cleavage of indoleglycerol phosphate that produces d-glyceraldehyde 3-phosphate and indole. The beta subunit catalyzes the formation of L-tryptophan from indole and serine. This reaction may use the indole created by the alpha subunit. The beta subunit is responsible for a pyridoxal phosphate (PLP)-dependent condensation of indole and L-serine into L-tryptophan.

The present disclosure provides a microorganism comprising an overexpressed tryptophan synthase. In some embodiments, the tryptophan synthase is endogenous and expression of the tryptophan synthase is increased by altering culture conditions. In some embodiments, the microorganism comprises an exogenous nucleic acid molecule that encodes an endogenous tryptophan synthase. In some embodiments, the microorganism comprises an exogenous nucleic acid molecule that encodes an exogenous tryptophan synthase, optionally the beta subunit of said tryptophan synthase. In some embodiments, the exogenous tryptophan synthase comprises an amino acid sequence with at least 80%, at least 85%, at least 90%, at least 95%, at least 99%, or 100% sequence identity to the sequence as shown in any of SEQ ID NOs: 19-22.

One or more metabolic pathways in a microorganism may be genetically modified to increase the endogenous production of tryptophan, thereby increasing the amount of the key precursor for the biosynthesis of tryptamine and subsequent biosynthesis of substituted tryptamines, by editing enzymes such as, for example, Aro1, Aro2, Aro3, Aro4, Trp1, Trp2, Trp3, Trp4, Trp5, Seri , Ser2, Ser3, GIn1 as disclosed in any of WO2019/180309, WO2021/110992, and WO2021/097452, the contents of which are incorporated herein by reference.

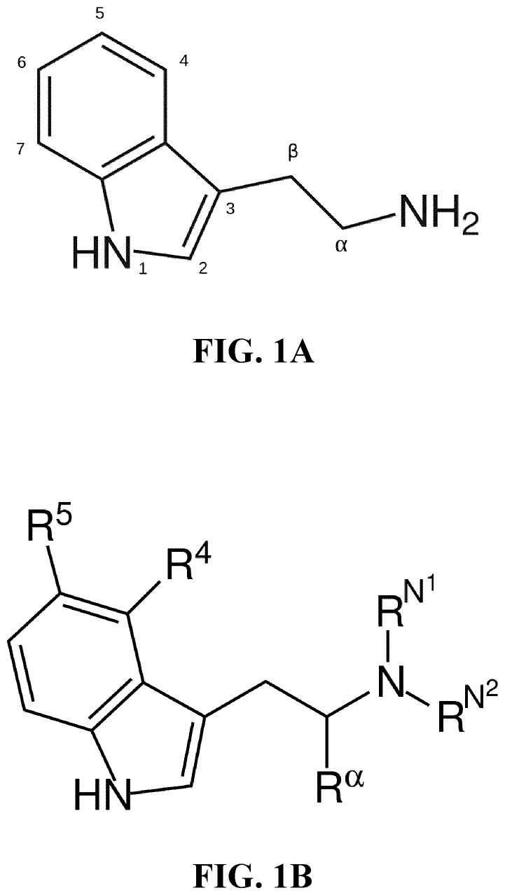

Tryptamine (FIG. 1) is a monoamine alkaloid comprising an indole ring joined to an amine group by an ethyl side chain at the 3-carbon of the pyrrole ring. Tryptamine may be synthesized by the decarboxylation of tryptophan. Decarboxylation of tryptophan into tryptamine may be performed enzymatically by the action of a tryptophan decarboxylase, such as the enzymes as shown in SEQ ID NOs: 1-3 and 23-35. Enzymatic decarboxylation of trypthophan can be performed in vitro by the incubation of tryptophan with a tryptophan decarboxylase, or in vivo by the endogenous or transgenic expression of a tryptophan decarboxylase in a microorganism to convert tryptophan into tryptamine. Decarboxylation of tryptophan into tryptamine may be performed by chemical decarboxylation or by thermolytic decarboxylation as known in the art (as disclosed for example in Laval and Golding, Synlett, 2003, 4:542-546, the contents of which are incorporated herein by reference). Tryptamine and substituted tryptamines may function in mammals as neurotransmitters and/or neuromodulators.

The present disclosure provides microorganisms capable of producing at least one substituted tryptamine. As used herein, the term “substituted tryptamine” refers to a molecule derived from tryptamine and may be used interchangeably with the term “tryptamine derivative”. In some embodiments, the substituted tryptamine comprises substitutions at one or more positions defined as 1, 2, 3, 4, 5, 6, 7, α, and/or β as shown in FIG. 1A or at one or more positions defined as Rα, R4, R5, RN1, and/or RN2 as shown in FIG. 1B. The term “substituted”, when used with an atom or group, refers to the designated atom or group where one or more hydrogen atoms on the atom or group is replaced with one or more substituents other than hydrogen, provided that the referred to atom or group's normal valence is not exceeded. A substituted tryptamine may be derived from tryptamine by substitution of a hydrogen for a functional group such as, but not limited to, an OH, an COOH, phosphate group, a methyl, a dimethyl allyl, or a halogen. A substituted tryptamine may be a molecule comprising an indole ring derived from tryptamine or a substituted tryptamine intermediate. In some embodiments, the substituted tryptamine is serotonin, N-acetyl serotonin, dimethylallyl tryptamine, lysergic acid diethylamide, N-methyltryptamine, N,N-Dimethyltryptamine, N,N,N-Trimethyltryptamine, N,N,N-Trimethyl-4-phosphoryloxytryptamine (aeruginascin), psilocybin, psilocin, baeocystin, norbaeocystin, 4-hydroxytryptamine, N-acetyl-4-hydroxytrptamine, gramine, clavine, indole-acetic acid, ateviridine, Pindolol, bufotenin, aurantioclavine, and/or a halogenated substituted tryptamine (e.g. a halogenated tryptamine, a dihalogenated tryptamine, a trihalogenated tryptamine, a halogenated N-methyltryptamine, a halogenated N,N-dimethyltryptamine, a halogenated N,N,N-trimethyltryptamine, a dihalogenated N-methyltryptamine, a dihalogenated N,N-dimethyltryptamine, a dihalogenated N,N,N-trimethyltryptamine, a trihalogenated N-methyltryptamine, a trihalogenated N,N-dimethyltryptamine or a trihalogenated N,N,N-trimethyltryptamine).

The present disclosure provides microorganisms comprising at least one nucleic acid molecule that encodes at least one substituted tryptamine biosynthetic pathway enzyme. As used herein, “substituted tryptamine biosynthetic pathway” refers to a biochemical pathway comprising one or more enzymatic steps that produces a substituted tryptamine. As used herein, “substituted tryptamine biosynthetic pathway enzyme” refers to an enzyme that produces tryptamine, a substituted tryptamine intermediate, or a substituted tryptamine by conversion of a substrate. A substituted tryptamine biosynthetic pathway may begin with the enzymatic conversion of L-tryptophan into tryptamine, with the enzymatic conversion of L-tryptophan into a substituted tryptamine intermediate, or the enzymatic conversion of L-tryptophan into a substituted tryptamine.

In some embodiments, the substituted tryptamine biosynthetic pathway is a biosynthetic pathway that is found to naturally occur in a microorganism. In some embodiments, the substituted tryptamine biosynthetic pathway recapitulates a biosynthetic pathway that is found to naturally occur in a microorganism but using analogous enzymes in place of the enzymes normally used in the naturally occurring pathway. In some embodiments, the substituted tryptamine biosynthetic pathway is a biosynthetic pathway that does not occur in nature.

### Microorganisms and Cell Culture

A microorganism may be genetically engineered to comprise at least one nucleic acid molecule encoding at least one substituted tryptamine biosynthetic pathway enzyme.

As used herein, the term “genetically engineered microorganism” refers to a microorganism whose genetic material has been altered using molecular biology techniques such as but not limited to molecular cloning, recombinant DNA methods, transformation and gene transfer. The genetically engineered microorganism includes a living modified microorganism, genetically modified microorganism or a transgenic microorganism. Genetic alteration includes addition, deletion, modification and/or mutation of genetic material. Such genetic engineering as described herein in the present disclosure may increase production of tryptophan, tryptamine, and/or a substituted tryptamine.

The term “nucleic acid molecule”, as used herein, is intended to include unmodified DNA or RNA or modified DNA or RNA. For example, it is useful for the nucleic acid molecules of the disclosure to be composed of single- and double-stranded DNA, DNA that is a mixture of single- and double-stranded regions, single- and double-stranded RNA, and RNA that is a mixture of single- and double-stranded regions, hybrid molecules comprising DNA and RNA that may be single-stranded or, more typically double-stranded or a mixture of single- and double-stranded regions. In addition, it is useful for the nucleic acid molecules to be composed of triple-stranded regions comprising RNA or DNA or both RNA and DNA. The nucleic acid molecules of the disclosure may also contain one or more modified bases or DNA or RNA backbones modified for stability or for other reasons. “Modified” bases include, for example, tritiated bases and unusual bases such as inosine. A variety of modifications can be made to DNA and RNA; thus “nucleic acid molecule” embraces chemically, enzymatically, or metabolically modified forms. The term “polynucleotide” shall have a corresponding meaning. In some embodiments, a genetically engineered microorganism comprises at least one nucleic acid molecule described herein.

As used herein, the term “exogenous” refers to an element that has been introduced into a cell. An exogenous element can include a protein or a nucleic acid. An exogenous nucleic acid is a nucleic acid that has been introduced into a cell, such as by a method of transformation. An exogenous nucleic acid may code for the expression of an RNA and/or a protein. An exogenous nucleic acid may have been derived from the same species (homologous) or from a different species (heterologous). An exogenous nucleic acid may comprise a homologous sequence that is altered such that it is introduced into the cell in a form that is not normally found in the cell in nature. For example, an exogenous nucleic acid that is homologous may contain mutations, being operably linked to a different control region, or being integrated into a different region of the genome, relative to the endogenous version of the nucleic acid. An exogenous nucleic acid may be incorporated into the chromosomes of the transformed cell in one or more copies, into the plastid or mitochondrial DNA of the transformed cell, or be maintained as a separate nucleic acid outside of the transformed cell genome.

The phrase “introducing a nucleic acid molecule into a microorganism” includes both the stable integration of the nucleic acid molecule into the genome of a microorganism to prepare a genetically engineered microorganism as well as the transient integration of the nucleic acid into microorganism. The introduction of a nucleic acid into a cell is also known in the art as transformation. The nucleic acid vectors may be introduced into the microorganism using techniques known in the art including, without limitation, agitation with glass beads, electroporation, agrobacterium-mediated transformation, an accelerated particle delivery method, i.e. particle bombardment, a cell fusion method or by any other method to deliver the nucleic acid vectors to a microorganism.

The term “nucleic acid sequence” as used herein refers to a sequence of nucleoside or nucleotide monomers consisting of naturally occurring bases, sugars and intersugar (backbone) linkages and includes cDNA. The term also includes modified or substituted sequences comprising non-naturally occurring monomers or portions thereof. The nucleic acid sequences of the present application may be deoxyribonucleic acid sequences (DNA) or ribonucleic acid sequences (RNA) and may include naturally occurring bases including adenine, guanine, cytosine, thymidine and uracil. The sequences may also contain modified bases. Examples of such modified bases include aza and deaza adenine, guanine, cytosine, thymidine and uracil; and xanthine and hypoxanthine. The nucleic acid can be either double stranded or single stranded, and represents the sense or antisense strand. Further, the term “nucleic acid” includes the complementary nucleic acid sequences.

A nucleic acid molecule may be incorporated into a vector. As used herein, the term “vector” or “nucleic acid vector” means a nucleic acid molecule, such as a plasmid, comprising regulatory elements and a site for introducing transgenic DNA (e.g. a nucleic acid molecule encoding at least one substituted tryptamine biosynthetic pathway enzyme), which is used to introduce said transgenic DNA into a microorganism. The transgenic DNA can encode a heterologous protein, which can be expressed in the microorganism. The transgenic DNA can be integrated into nuclear, mitochondrial or chloroplastic genomes through homologous or non-homologous recombination. The transgenic DNA can also replicate without integrating into nuclear, mitochondrial or chloroplastic genomes in an extra-chromosomal vector. The vector can contain a single, operably-linked set of regulatory elements that includes a promoter, a 5′ untranslated region (5′ UTR), an insertion site for transgenic DNA, a 3′ untranslated region (3′ UTR) and a terminator sequence. Vectors useful in the present methods are well known in the art. In one embodiment, the nucleic acid molecule is an episomal vector.

As used herein, the term “episomal vector” refers to a DNA vector based on a bacterial episome that can be expressed in a transformed cell without integration into the transformed cell genome by staying extrachromosomal. Episomal vectors can be transferred from a bacteria (e,g, Escherichia col') to another target microorganism (e.g. a microalgae) via conjugation or by purification and mechanical introduction such as electroporation.

In another embodiment, the vector is a commercially available vector. As used herein, the term “expression cassette” means a single, operably-linked set of regulatory elements that includes a promoter, a 5′ untranslated region (5′ UTR), an insertion site for transgenic DNA, a 3′ untranslated region (3′ UTR) and a terminator sequence. In an embodiment, the at least one nucleic acid molecule is an episomal vector.

The term “operably-linked”, as used herein, refers to an arrangement of two or more components, wherein the components so described are in a relationship permitting them to function in a coordinated manner. For example, a transcriptional regulatory sequence or a promoter is operably-linked to a coding sequence if the transcriptional regulatory sequence or promoter facilitates aspects of the transcription of the coding sequence. The skilled person can readily recognize aspects of the transcription process, which include, but not limited to, initiation, elongation, attenuation and termination. In general, an operably-linked transcriptional regulatory sequence joined in cis with the coding sequence, but it is not necessarily directly adjacent to it.

Vectors encoding at least one substituted tryptamine biosynthetic pathway enzyme may contain elements suitable for the proper expression of the enzyme in the microorganism. Specifically, each expression vector contains a promoter that promotes transcription in microorganisms. The term “promoter,” as used herein, refers to a nucleotide sequence that directs the transcription of a gene or coding sequence to which it is operably-linked. The skilled person can readily appreciate inducible promoters including chemically-inducible promoters, alcohol inducible promoters, and estrogen inducible promoters can also be used. Predicted promoters, such as those that can be found from genome database mining may also be used. In addition, the nucleic acid molecule or vector may contain one or more introns in front of the cloning site or within a gene sequence to drive a strong expression of the gene of interest. Selectable marker genes can also be linked on the vector, such as the kanamycin resistance gene (also known as neomycin phosphotransferase gene II, or nptII), zeocin resistance gene, hygromycin resistance gene, Basta resistance gene, hygromycin resistance gene, or others.

Nucleic acid sequences encoding substituted tryptamine biosynthetic pathway enzymes as described herein can be provided in vectors in different arrangements or combinations. Each individual sequence that encodes an enzyme of a cannabinoid biosynthetic pathway can be provided in separate vectors. Alternatively, multiple sequences can be provided together in the same vector.

Where more than one sequence that encodes an enzyme is provided in the same vector, the sequences can be provided in separate expression cassettes, or together in the same expression cassette. Where two or more sequences are in the same expression cassette, they can be provided in the same open reading frame so as to produce a fusion protein. Two or more sequences that encode a fusion protein can be separated by linker sequences that encode restriction nuclease recognition sites or self-cleaving peptide linkers. Accordingly, a microorganism can be engineered by stepwise transfection with multiple vectors that each comprises nucleic acid molecules that encode one or more enzymes of a substituted tryptamine biosynthetic pathway, or with a single vector that comprises nucleic acid molecules that encode all of the enzymes of a substituted tryptamine biosynthetic pathway.

Microorganisms may be cultured in conditions that are permissive to their growth. It is known that photosynthetic microorganisms such as microalgae and cyanobacteria are capable of carbon fixation wherein carbon dioxide (which is not a fixed carbon source) is fixed into organic molecules such as sugars using energy from a light source. The fixation of carbon dioxide using energy from a light source is photosynthesis. Suitable sources of light for the provision of energy in photosynthesis include sunlight and artificial lights. Photosynthetic microorganisms are capable of growth and/or metabolism without a fixed carbon source. Photosynthetic microorganisms can fix carbon dioxide from a variety of sources, including atmospheric carbon dioxide, industrially-discharged carbon dioxide (e.g. flue gas and flaring gas), and from soluble carbonates (e.g. NaHCO3 and Na2CO3). A non-fixed carbon source such as carbon dioxide can be added to a culture of microalgae by injection or by bubbling of a carbon dioxide gas mixture into the culture medium. Photosynthetic growth is a form of autotrophic growth, wherein a microorganism is able to produce organic molecules on its own using an external energy source such as light. This is in contrast to heterotrophic growth, wherein a microorganism must consume organic molecules for growth and/or metabolism. Heterotrophic microorganisms therefore require a fixed carbon source for growth and/or metabolism. Some photosynthetic microorganisms are capable of mixotrophic growth, wherein the microorganism fixes carbon by photosynthesis while also consuming fixed carbon sources. In mixotrophic growth, the autotrophic metabolism is integrated with a heterotrophic metabolism that oxidizes reduced carbon sources available in the culture medium. Photosynthetic microorganisms are commonly cultivated in mixotrophic conditions by adding fixed carbon sources as described herein to the culture medium. Common sources of fixed carbon that are used include glucose, ethanol, or waste products from industry such as acetate or glycerol. Microorganisms such as microalgae and cyanobacteria may be cultured using methods and conditions known in the art. Some microorganisms are capable of chemoautotrophic growth, Similar to photosynthetic microorganisms, chemoautotrophic organisms are capable of carbon dioxide fixation but using energy derived from chemical sources (e.g. hydrogen sulfide, ferrous iron, molecular hydrogen, ammonia) rather than light.

In some embodiments, culture conditions of a microorganism may be altered to induce overexpression of a tryptophan synthase in the microorganism. In some embodiments, culture conditions of a microorganism may be altered to induce overexpression of an endogenous tryptophan synthase in the microorganism. In some embodiments, the altered culture conditions comprise nutrient limitation (e.g. phosphate deprivation, nitrogen deprivation, iron deprivation) and/or light deprivation (e.g. withdrawal of light sources, switching of light source spectra).

As used herein, “overexpression” refers to elevated expression of a gene or polypeptide in a genetically engineered microorganism compared to a corresponding wild-type microorganism, or to elevated expression of a gene or polypeptide in a microorganism cultured in altered conditions compared to a corresponding microorganism cultured under normal or control conditions.

In some embodiments, the microorganism may be a microalga, a stramenopile, a cyanobacterium, a bacterium, a protist, or a fungus.

In some embodiments, the microalga is a species from Chlorophyceae, Trebouxiophyxeae, Coscinodiscophyceae, Bacillariphyceae, Eustigmatophyceae, or Labyrinthylomycetes. In some embodiments the microalga is a species from Chlamydomonales, Chlorellales, Thalassiosirales, Baccilariales, Eustigmatales, or Labyrinthulales. In some embodiments, the microalga is a species from Acutodesmus, Ankistrodesmus, Asteromonas, Aurantiochytrium, Auxenochlorella, Basichlamys, Botryococcus, Botryokoryne, Borodinella, Brachiomonas, Catena, Carteria, Chaetophora, Characiochloris, Characiosiphon, Chlainomonas, Chlamydomonas, Chlorella, Chlorochytrium, Chlorococcum, Chlorogonium, Chloromonas, Closteriopsis, Dictyochloropsis, Dunaliella, Ellipsoidon, Eremosphaera, Eudorina, Floydiella, Friedmania, Haematococcus, Hafniomonas, Heterochlorella, Gonium, Halosarcinochlamys, Koliella, Lobocharacium, Lobochlamys, Lobomonas, Lobosphaera, Lobosphaeropsis, Marvania, Monoraphidium, Myrmecia, Nannochloris, Nannochloropsis, Oocystis, Oogamochlamys, Pabia, Pandorina, Parietochloris, Phacotus, Phaeodactylum, Platydorina, Platymonas, Pleodorina, Polulichloris, Polytoma, Polytomella, Prasiola, Prasiolopsis, Prasiococcus, Prototheca, Pseudochlorella, Pseudocarteria, Pseudotrebouxia, Pteromonas, Pyrobotrys, Rosenvingiella, Scenedesmus, Schizochytrium, Spirogyra, Stephanosphaera, Tetrabaena, Tetraedron, Tetraselmis, Thalassiosira, Thraustochytium, Trebouxia, Trochisciopsis, Ulkenia, Viridiella, Vitreochlamys, Volvox, Volvulina, Vulcanochloris, Watanabea, Yamagishiella, Euglena, lsochrysis, or Nannochloropsis. In an embodiment, the microalga is Chlamydomonas reinhardtii, Chlorella vulgaris, Chlorella sorokiniana, Chlorella protothecoides, Tetraselmis chuff, Nannochloropsis oculata, Phaeodactylum tricornutum, Thalassiosira pseudonana, Prototheca moriformis, Scenedesmus obliquus, Acutodesmus dimorphus, Schizochytrium limacinum, Dunaliella tertiolecta, Aurantiochytrium sp., Thraustochytrium sp., Ulkenia sp., or Haematococus plucialis. In another embodiment, the microalga is a diatom, optionally Phaeodactylum tricornutum or Thalassiosira pseudonana.

In some embodiments, the stramenopile is a species from Coscinodiscophyceae, Bacillariphyceae, Eustigmatophyceae, or Labyrinthylomycetes. In some embodiments, the stramenopile is a species from Thalassiosirales, Baccilariales, Eustigmatales, or Labyrinthulales. In some embodiments, the stramenoplie is a species from Thalassiosira, Phaeodactylum, Nannochloropsis, Schizochytrium Aurantiochytrium, Thraustochytrium, or Ulkenia. In an embodiment, the stramenopile is Nannochloropsis oculata, Phaeodactylum tricornutum, Thalassiosira pseudonana, Schizochytrium limacinum, Schizochytrium sp., Aurantiochytrium sp., Thraustochytrium sp., or Ulkenia sp.

In some embodiments, the cyanobacterium is from Spirulinaceae, Phormidiaceae, Synechococcaceae, or Nostocaceae. In an embodiment, the cyanobacterium is Arthrospira plantesis, Arthrospira maxima, Synechococcus elongatus, or Aphanizomenon flos-aquae.

In some embodiments, the microorganism is a bacterium, for example from the genera Escherichia, Bacillus, Caulobacter, Mycoplasma, Pseudomonas, Streptomyces, or Zymomonas.

In some embodiments, the microorganism is a protist, for example from the genera Dictyostelium, Tetrahymena, Emiliania.

In some embodiments, the microorganism is a fungus, for example from the genera Aspergillus, Saccharomyces, Schizosaccharomyces, or Fusarium.

In some embodiments, the microorganism is a microalga. Without being bound by theory, it is believed that microalgae possess advantageous properties for the biosynthesis of substituted tryptamines. Compared to yeast and bacteria, microalgae contain higher concentrations of cofactors needed for tryptophan decarboxylase and tryptamine monooxygenase activity such as pyridoxal phosphate (PLP) (De Roeck-Holtzhauer et al., J Appl Phycol, 1991, 3:259-264; Dempsey et al., J. Bacteriol., 1971, 108(1):415-421). Further, compared to yeast and bacteria, the cytosol of microalgae is a less oxidative environment that improves the activity of enzymes such as N-methyltransferases and indole-ethylamine methyltransferases that catalyze the transfer of methyl groups to produce substituted tryptamines such as, for example, psilocybin, psilocyn, bufotenine, aeruginascin, and N,N-dimethyltryptamine.

In some embodiments, the microorganism is a microalga or a stramenopile. It was surprisingly discovered by the present inventors that microalgae and stramenopiles are tolerant of high concentrations of tryptamine that kill or severely restrict growth of yeast and bacteria. Therefore, microalgae and stramenopiles are suitable for high-yield biosynthesis of substituted tryptamines in culture with high concentrations of supplemented tryptamine.

Tryptamine is the key precursor of substituted tryptamine biosynthetic pathways. The in vivo synthesis of tryptophan and/or the in vivo synthesis of tryptamine by enzymatic decarboxylation of tryptophan may be rate-limiting steps that limit the final yield of biosynthetically-produced substituted tryptamines in microorganisms. In some embodiments, microorganisms comprising at least one nucleic acid molecule encoding at least one substituted tryptamine biosynthetic pathway enzyme may be cultured in media supplemented with tryptamine to increase the yield of a substituted tryptamine. Further, supplementation with tryptamine may obviate the need to overexpress an endogenous trypthophan decarboxylase or to introduce an exogenous tryptophan decarboxylase into a microorganism comprising at least one nucleic acid molecule encoding at least one substituted tryptamine biosynthetic pathway enzyme, reducing the amount of genetic modification required. In some embodiments, microorganisms comprising at least one nucleic acid molecule encoding at least one substituted tryptamine biosynthetic pathway enzyme may be cultured in media with a high concentration of tryptamine for the high-yield biosynthesis of substituted tryptamines.

In some embodiments, there is provided a cell culture comprising (i) a microalga comprising at least one nucleic acid molecule that encodes at least one substituted tryptamine biosynthetic pathway enzyme and (ii) a culture media supplemented with a high concentration of tryptamine. In some embodiments, there is provided a cell culture comprising (i) a stramenopile comprising at least one nucleic acid molecule that encodes at least one substituted tryptamine biosynthetic pathway enzyme and (ii) a culture media supplemented with a high concentration of tryptamine.

High concentrations of tryptamine may be toxic to microorganisms, reducing their viability and growth rates and subsequently the yield of substituted tryptamine. In some embodiments, a high concentration of supplemented tryptamine is at least about 1 mM, 2 mM, 3 mM, 4 mM, 5 mM, 6 mM, 7 mM, 9 mM, or 10 mM of tryptamine in the culture media. In some embodiments, microorganisms comprising at least one nucleic acid molecule encoding at least one substituted tryptamine biosynthetic pathway enzyme must be tolerant of high concentrations of tryptamine.

In some embodiments, microorganisms that are tolerant of high concentrations of tryptamine may maintain at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, and least 95%, or at least 99% cell viability when cultured with a high concentration of supplemented tryptamine during the log phase and/or the stationary phase, for example, the log phase. In some embodiments, microorganisms that are tolerant of high concentrations of tryptamine may maintain at least 75%, at least 80%, at least 85%, at least 90%, at least 95%, at least 99%, or 100% of the growth rate when cultured with a high concentration of supplemented tryptamine relative to the growth rate when cultured without supplemented tryptamine during the log phase and/or the stationary phase, for example, the log phase.

In some embodiments, a microorganism that is tolerant of high concentrations of tryptamine is a microalga. In some embodiments, a microorganism that is tolerant of high concentrations of tryptamine is a stramenopile. In some embodiments, a microorganism that is tolerant of high concentrations of tryptamine is a species of Bacillariophyceae, Eustigmatophyceae, or Labyrinthulomycetes. In some embodiments, a microorganism that is tolerant of high concentrations of tryptamine is Chlamydomonas reinhardtii, Chlorella vulgaris, Chlorella sorokiniana, Chlorella protothecoides, Tetraselmis chuff, Nannochloropsis australis, Nannochloropsis gaditana, Nannochloropsis granulata, Nannochloropsis limnetica, Nannochloropsis oceanica, Nannochloropsis oculata, Nannochloropsis salina, Phaeodactylum tricornutum, Thalassiosira pseudonana, Prototheca moriformis or Schizochytrium limacinum.

Many media for the culture of microorganisms are known in the art and which are suitable for use in culturing the microorganisms described herein including, but not limited to, F/2 media, L1 media, Tris acetate phosphate (TAP), or Bold's Basal Medium (BBM), as disclosed in de Carvalho et al., Biofuels from Algae (Second Edition), Elsevier, 2019, 33-50, the contents of which are incorporated herein by reference.

### Substituted Tryptamine Biosynthetic Pathways

In some embodiments, the substituted tryptamine is psilocybin. Psilocybin is a substituted tryptamine (FIG. 1A) wherein Rα=H, R4=PO₄, R5=H, RN1=CH₃, RN2=CH₃. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, and an N-methyltransferase. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, and an N-methyltransferase, and wherein the microorganism does not comprise an exogenous nucleic acid molecule that encodes a tryptophan decarboxylase.

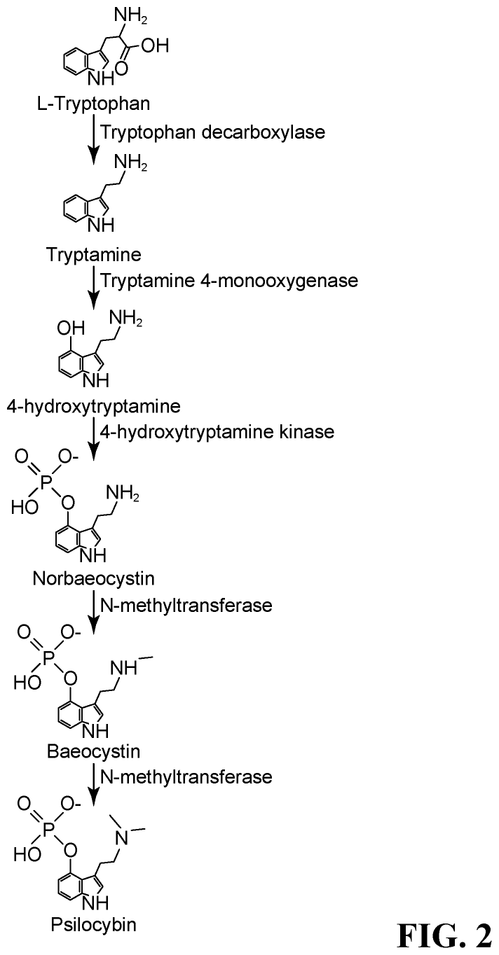

An exemplary substituted tryptamine biosynthetic pathway for psilocybin is shown in FIG. 2. L-tryptophan is decarboxylated by the activity of a tryptophan decarboxylase to form tryptamine. An OH group is transferred to the 4-carbon by the activity of a tryptamine 4-monooxygenase to form 4-hydroxytryptamine. A phosphate group is transferred to 4-hydroxytryptamine by the activity of a 4-hydroxytryptamine kinase to form norbaeocystin. Iterative methyl transfer to the amine group of norbaeocystin by the activity of an N-methyltransferase forms the monomethylated intermediate baeocystin and then psilocybin. Dephosphorylation of psilocybin either spontaneously or by the action of a phosphatase produces psilocin. Phosphorylation of psilocin by a 4-hydroxytryptamine kinase again produces psilocin. When tryptamine is already present (e.g. due to supplementation) the substituted tryptamine biosynthetic pathway for psilocybin does not require a tryptophan decarboxylase and begins with the activity of a tryptamine 4-monooxygenase.

A tryptophan decarboxylase is a substituted tryptamine biosynthetic pathway enzyme capable of removing the carboxyl from L-tryptophan to produce tryptamine. In some embodiments, the tryptophan decarboxylase is a polypeptide comprising an amino acid sequence with at least 80%, at least 85%, at least 90%, at least 95%, at least 99%, or 100% sequence identity to the sequence as shown in any of SEQ ID NOs: 1-3 and 23-35.

A tryptamine 4-monooxygenase is a substituted tryptamine biosynthetic pathway enzyme capable of transferring an OH group to the 4-carbon of tryptamine. In some embodiments, the tryptamine 4-monooxygenase is a polypeptide comprising an amino acid sequence with at least 80%, at least 85%, at least 90%, at least 95%, at least 99%, or 100% sequence identity to the sequence as shown in any of SEQ ID NOs: 4-7 and 36-42. The activity of a tryptamine 4-monooxygenase in a microorganism as described herein may be enhanced by co-expression with a cytochrome p450 reductase as shown in SEQ ID NO: 108.

A 4-hydroxytryptamine kinase is a substituted tryptamine biosynthetic pathway enzyme capable of 4-0 phosphorylation of 4-hydroxytryptamine. In some embodiments, the 4-hydroxytryptamine kinase is a polypeptide comprising an amino acid sequence with at least 80%, at least 85%, at least 90%, at least 95%, at least 99%, or 100% sequence identity to the sequence as shown in any of SEQ ID NOs: 8-12 and 43-59.

An N-methyltransferase is a substituted tryptamine biosynthetic pathway enzyme capable of iterative methyl transfer to the amine group of norbaeocsytin/baeocystin. In some embodiments, the N-methyltransferase is a polypeptide comprising an amino acid sequence with at least 80%, at least 85%, at least 90%, at least 95%, at least 99%, or 100% sequence identity to the sequence as shown in any of SEQ ID NOs: 13-14 and 109-321.

In some embodiments, a microorganism for use in the production of psilocybin comprises at least one nucleic acid molecule encoding a tryptophan decarboxylase as shown in any of SEQ ID NOs: 1-3 and 23-35, a tryptamine 4-monooxygenase as shown in any of SEQ ID NOs: 4-7 and 39-42, a 4-hydroxytryptamine kinase as shown in any of SEQ ID NOs: 8-12 and 43-59, and an N-methyltransferase as shown in any of SEQ ID NOs: 13-14 and 109-321, or a polypeptide at least 80% identical to any thereof. When tryptamine is already present (e.g. due to supplementation) the at least one nucleic acid molecule may exclude a trypthophan decarboxylase.

In some embodiments, the substituted tryptamine is serotonin. Serotonin is a substituted tryptamine (FIG. 1A) wherein Rα=H, R4=H, R5=OH, RN1=H, RN2=H. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase and a tryptamine 5-hydroxylase. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises tryptamine 5-hydroxylase, and wherein the microorganism does not comprise an exogenous nucleic acid molecule that encodes a tryptophan decarboxylase.

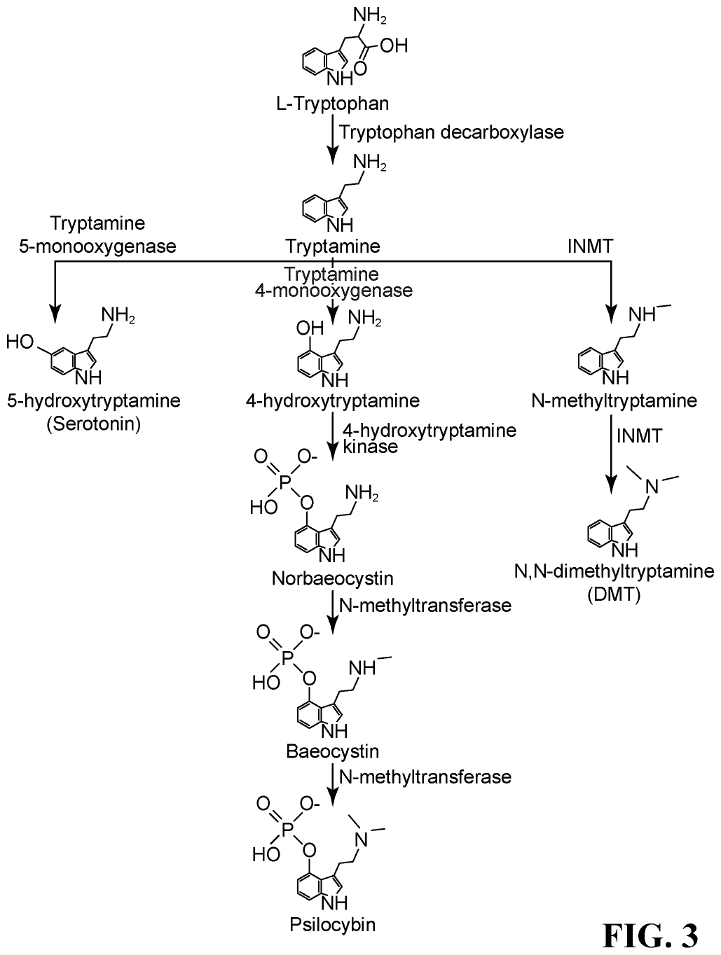

An exemplary substituted tryptamine biosynthetic pathway for serotonin is shown in FIG. 3 (left pathway). L-tryptophan is decarboxylated by the activity of a tryptophan decarboxylase to form tryptamine. An OH group is transferred to the 5-carbon by the activity of a tryptamine 5-hydroxylase to form serotonin. When tryptamine is already present (e.g. due to supplementation) the substituted tryptamine biosynthetic pathway for serotonin does not require a tryptophan decarboxylase and begins with the activity of a tryptamine 5-hydroxylase.

A tryptamine 5-hydroxylase (also known as a tryptamine 5-monooxygenase) is a substituted tryptamine biosynthetic pathway enzyme capable of transferring an OH group to the 5-carbon of tryptamine. In some embodiments, the tryptamine 5-hydroxylase is a polypeptide comprising an amino acid sequence with at least 80%, at least 85%, at least 90%, at least 95%, at least 99%, or 100% sequence identity to the sequence as shown in SEQ ID NO: 15.

In some embodiments, the substituted tryptamine is N,N-dimethyltryptamine (DMT). DMT is a substituted tryptamine (FIG. 1A) wherein Rα=H, R4=H, R5=H, RN1=CH3, RN2=CH₃. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase and an indole-ethylamine methyltransferase (INMT). In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises an indole-ethylamine methyltransferase (INMT), and wherein the microorganism does not comprise an exogenous nucleic acid molecule that encodes a tryptophan decarboxylase.

An exemplary substituted tryptamine biosynthetic pathway for DMT is shown in FIG. 3 (right pathway). L-tryptophan is decarboxylated by the activity of a tryptophan decarboxylase to form tryptamine. Iterative methyl transfer to the amine group of tryptamine by the activity of an indole-ethylamine methyltransferase forms the monomethylated intermediate N-methyltryptamine and then DMT. When tryptamine is already present (e.g. due to supplementation) the substituted tryptamine biosynthetic pathway for DMT does not require a tryptophan decarboxylase and begins with the activity of an INMT.

An indole-ethylamine methyltransferase is a substituted tryptamine biosynthetic pathway enzyme capable of iterative methyl transfer to the amine group of tryptamine/N-methyltryptamine. In some embodiments, the indole-ethylamine methyltransferase is a polypeptide comprising an amino acid sequence with at least 80%, at least 85%, at least 90%, at least 95%, at least 99%, or 100% sequence identity to the sequence as shown in any of SEQ ID NOs: 16 and 60-98.

In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, an N-methyltransferase, and a tryptamine 5-hydroxylase, and is capable of producing psilocybin and/or serotonin. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, an N-methyltransferase, and a tryptamine 5-hydroxylase, and is capable of producing psilocybin and/or serotonin, and wherein the microorganism does not comprise an exogenous nucleic acid molecule that encodes a tryptophan decarboxylase.

In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, an N-methyltransferase, and an indole-ethylamine methyltransferase (INMT), and is capable of producing psilocybin and/or DMT. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, an N-methyltransferase, and an indole-ethylamine methyltransferase (INMT), and is capable of producing psilocybin and/or DMT, and wherein the microorganism does not comprise an exogenous nucleic acid molecule that encodes a tryptophan decarboxylase.

In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, an N-methyltransferase, a tryptamine 5-hydroxylase, and an indole-ethylamine methyltransferase (INMT), and is capable of producing psilocybin, serotonin and/or DMT. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, an N-methyltransferase, a tryptamine 5-hydroxylase, and an indole-ethylamine methyltransferase (INMT), and is capable of producing psilocybin, serotonin and/or DMT, and wherein the microorganism does not comprise an exogenous nucleic acid molecule that encodes a tryptophan decarboxylase.

In some embodiments, the substituted tryptamine is psilocybin. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, an indole-ethylamine methyltransferase (INMT), a tryptamine 4-monooxygenase, and a 4-hydroxytryptamine kinase. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises an indole-ethylamine methyltransferase (INMT), a tryptamine 4-monooxygenase, and a 4-hydroxytryptamine kinase, and wherein the microorganism does not comprise an exogenous nucleic acid molecule that encodes a tryptophan decarboxylase.

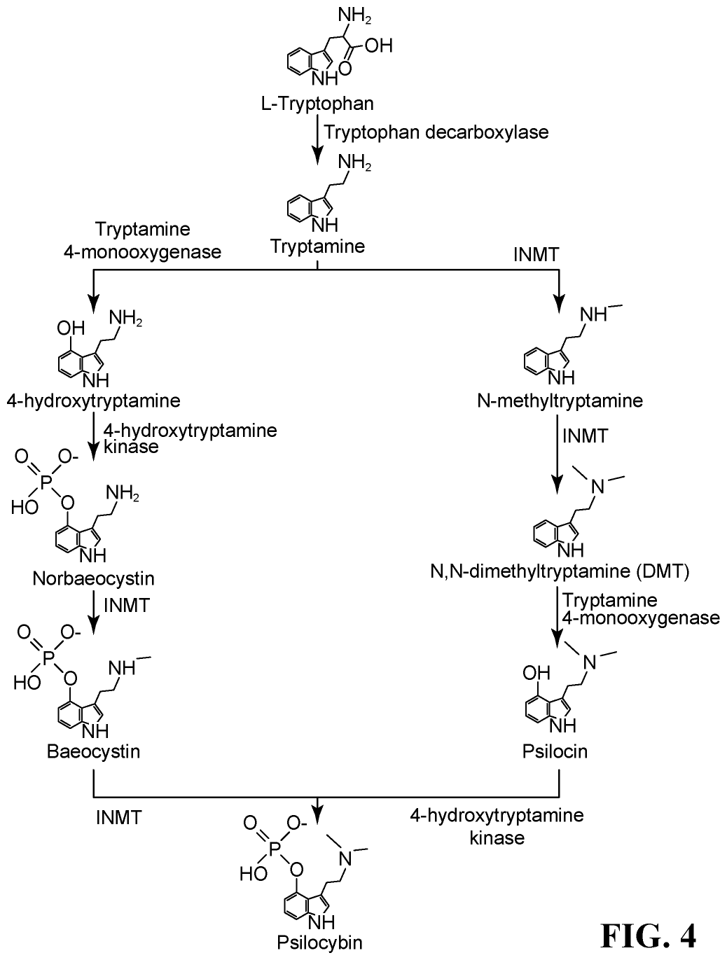

Exemplary substituted tryptamine biosynthetic pathways comprising indole-ethylamine methyltransferase for the production of psilocybin are shown in FIG. 4. In one pathway (FIG. 4, right), L-tryptophan is decarboxylated by the activity of a tryptophan decarboxylase to form tryptamine. Iterative methyl transfer to the amine group of tryptamine by the activity of an indole-ethylamine methyltransferase forms the monomethylated intermediate N-methyltryptamine and then DMT. An OH group is transferred to the 4-carbon of DMT by the activity of a tryptamine 4-monooxygenase to form psilocin. A phosphate group is transferred to psilocin by the activity of a 4-hydroxytryptamine kinase to form psilocybin. In another pathway (FIG. 4, left), L-tryptophan is decarboxylated by the activity of a tryptophan decarboxylase to form tryptamine. An OH group is transferred to the 4-carbon by the activity of a tryptamine 4-monooxygenase to form 4-hydroxytryptamine. A phosphate group is transferred to 4-hydroxytryptamine by the activity of a 4-hydroxytryptamine kinase to form norbaeocystin. Iterative methyl transfer to the amine group of norbaeocystin by the activity of an indole-ethylamine methyltransferase forms the monomethylated intermediate baeocystin and then psilocybin. When tryptamine is already present (e.g. due to supplementation) the substituted tryptamine biosynthetic pathways comprising indole-ethylamine methyltransferase for the production of psilocybin do not require a tryptophan decarboxylase and begins with the activity of an INMT or tryptamine 4-monooxygenase.

In some embodiments, the substituted tryptamine is dimethylallyl tryptamine. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a dimethylallyl tryptamine synthase. Dimethylallyl tryptamine is a useful precursor for the synthesis of indole-containing molecules such as lysergic acid dimethylamide (LSD). L-tryptophan is converted to dimethylallyl-tryptamine by the activity of a dimethylallyl tryptamine synthase. Dimethylallyl-tryptamine is a substituted tryptamine (FIG. 1A) wherein Rα=COOH, R4=dimethyl allyl, R5=H, RN1=H, RN2=H.

A dimethylallyl tryptamine synthase is a substituted tryptamine biosynthetic pathway enzyme capable of converting L-tryptophan into dimethylallyl-tryptamine. In some embodiments, the dimethylallyl tryptamine synthase is a polypeptide comprising an amino acid sequence with at least 80%, at least 85%, at least 90%, at least 95%, at least 99%, or 100% sequence identity to the sequence as shown in SEQ ID NO: 17.

In some embodiments, the substituted tryptamine is aurantioclavine. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a dimethylallyl tryptamine synthase and an aurantioclavine synthase.

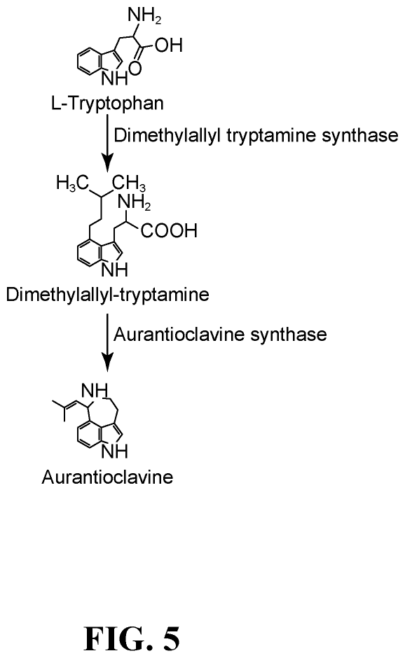

An exemplary substituted tryptamine biosynthetic pathway for aurantioclavine is shown in FIG. 5. L-tryptophan is converted to dimethylallyl-tryptamine by the activity of a dimethylallyl tryptamine synthase. Dimethylallyl-tryptamine is converted to aurantioclavine by the activity of aurantioclavine synthase.

An aurantioclavine synthase is a substituted tryptamine biosynthetic pathway enzyme capable of converting dimethylallyl tryptamine into aurantioclavine. In some embodiments, the aurantioclavine synthase is a polypeptide comprising an amino acid sequence with at least 80%, at least 85%, at least 90%, at least 95%, at least 99%, or 100% sequence identity to the sequence as shown in SEQ ID NO: 18.

In some embodiments, the substituted tryptamine is bufotenin (5-hydroxy-N,N-dimethyltryptamine). In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 5-hydroxylase, and an INMT. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 5-hydroxylase and an INMT, and wherein the microorganism does not comprise an exogenous nucleic acid molecule that encodes a tryptophan decarboxylase.

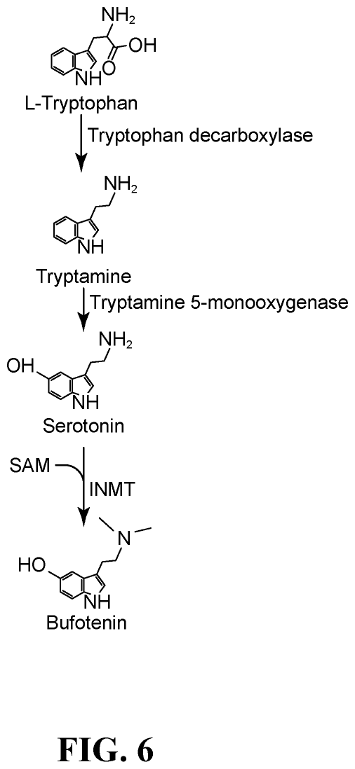

An exemplary substituted tryptamine biosynthetic pathway for bufotenin is shown in FIG. 6. L-tryptophan is decarboxylated by the activity of a tryptophan decarboxylase to form tryptamine. An OH group is transferred to the 5-carbon by the activity of a tryptamine 5-hydroxylase to form serotonin. Iterative methyl transfer to the amine group of serotonin by the activity of an indole-ethylamine methyltransferase forms bufotenin. When tryptamine is already present (e.g. due to supplementation) the substituted tryptamine biosynthetic pathway for bufotenindoes not require a tryptophan decarboxylase and begins with the activity of a tryptamine 5-hydroxylase.

In some embodiments, the substituted tryptamine is aeruginascin (N,N,N-trimethyl-4-phosphoryloxytryptamine). In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, and either an N-methyltransferase or an indole-ethylamine methyltransferase , wherein the N-methyltransferase or the indole-ethylamine methyltransferase is overexpressed and/or present in multiple copies. In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies, and wherein the microorganism does not comprise an exogenous nucleic acid molecule that encodes a tryptophan decarboxylase.

In some embodiments, the substituted tryptamine is a halogenated substituted tryptamine (e.g. a halogenated tryptamine, a dihalogenated tryptamine, a trihalogenated tryptamine, a halogenated N-methyltryptamine, a halogenated N,N-dimethyltryptamine, a halogenated N,N,N-trimethyltryptamine, a dihalogenated N-methyltryptamine, a dihalogenated N,N-dimethyltryptamine, a dihalogenated N,N,N-trimethyltryptamine, a trihalogenated N-methyltryptamine, a trihalogenated N,N-dimethyltryptamine or a trihalogenated N,N,N-trimethyltryptamine). In some embodiments, a microorganism provided herein comprises at least one nucleic acid molecule that encodes at least one substituted tryptamine biosynthetic pathway enzyme, wherein the at least one substituted tryptamine biosynthetic pathway enzyme comprises a tryptophan halogenase and a tryptophan decarboxylase.

A tryptophan halogenase is a substituted tryptamine biosynthetic pathway enzyme capable of transferring a halogen group onto L-tryptophan. Tryptophan halogenases may be regioselective, transferring the halogen group to a specific carbon on the indole of tryptophan. A tryptophan halogenase may be a tryptophan-2-halogenase, a tryptophan-5-halogenase, a tryptophan-6-halogenase, or a tryptophan-7-halogenase that transfers a halogen group to the 2, 5, 6, or 7 carbon (as shown in FIG. 1A) of the indole of L-tryptophan. In some embodiments, tryptophan halogenase is a polypeptide comprising an amino acid sequence with at least 80%, at least 85%, at least 90%, at least 95%, at least 99%, or 100% sequence identity to the sequence as shown in any of SEQ ID NOs: 99-106. The activity of a tryptophan halogenase may be enhanced by co-expression in a microorganism as described herein with a flavin reductase as shown in SEQ ID NO: 107.

In a biosynthetic pathway for a halogenated substituted tryptamine, a tryptophan halogenase catalyzes the transfer of a halogen group (e.g. a fluorine, a chlorine, a bromine, or an iodine) onto L-tryptophan to produce a halogenated L-tryptophan. Mono-, di-, or tri-halogenated trytophan may be produced by using one, two, or three separate regioselective tryptophan halogenases. Decarboxylation of the halogenated tryptophan by a tryptophan decarboxylase produces a halogenated tryptamine. The halogenated tryptamine may further be acted upon by other tryptamine biosynthetic pathway enzymes as disclosed herein (as they would act upon tryptamine) to produce downstream halogenated substituted tryptamines (e.g. a halogenated N-methyltryptamine, a halogenated N,N-dimethyltryptamine, a halogenated N,N,N-trimethyltryptamine, a dihalogenated N-methyltryptamine, a dihalogenated N,N-dimethyltryptamine, a dihalogenated N,N,N-trimethyltryptamine, a trihalogenated N-methyltryptamine, a trihalogenated N,N-dimethyltryptamine or a trihalogenated N,N,N-trimethyltryptamine).

In some embodiments, a microorganism provided herein comprising at least one nucleic acid molecule that encodes at least one substituted tryptamine biosynthetic pathway enzyme may further comprise at least one nucleic acid molecule that encodes an endogenous or an exogenous tryptophan synthase as described herein, optionally the beta subunit of said tryptophan synthase. In some embodiments, a microorganism provided herein comprising at least one nucleic acid molecule that encodes at least one substituted tryptamine biosynthetic pathway enzyme may be cultured in altered culture conditions as described herein to overexpress an endogenous tryptophan synthase.

Particular embodiments of the disclosure include, without limitation, the following:
1. A microorganism comprising an overexpressed tryptophan synthase.
2. The microorganism of embodiment 1, wherein the tryptophan synthase is endogenous and expression of the tryptophan synthase is increased by altering culture conditions.
3. The microorganism of embodiment 2, wherein the altered culture conditions comprise nutrient limitation (e.g. phosphate deprivation, nitrogen deprivation, iron deprivation) and/or light deprivation (e.g. withdrawal of light sources, switching of light source spectra).
4. The microorganism of any one of embodiments 1 to 3, comprising an exogenous nucleic acid molecule that encodes the endogenous tryptophan synthase.
5. The microorganism of embodiment 4, wherein the endogenous tryptophan synthase encoded by the exogenous nucleic acid molecule comprises an amino acid sequence with at least 80%, at least 85%, at least 90%, at least 95%, at least 99%, or 100% sequence identity to the wild-type endogenous tryptophan synthase.
6. The microorganism of any one of embodiments 1 to 3, comprising an exogenous nucleic acid molecule that encodes an exogenous tryptophan synthase.
7. The microorganism of embodiment 6, wherein the exogenous tryptophan synthase comprises an amino acid sequence with at least 80%, at least 85%, at least 90%, at least 95%, at least 99%, or 100% sequence identity to the sequence as shown in any of SEQ ID NOs: 19-22.
8. The microorganism of any one of embodiments 1 to 7, comprising at least one nucleic acid molecule that encodes at least one substituted tryptamine biosynthetic pathway enzyme.
9. The microorganism of embodiment 8, wherein the at least one substituted tryptamine biosynthetic pathway enzyme comprises one or more of a tryptophan decarboxylase, a tryptamine 4-monooxygenase, a 4-hydroxytryptamine kinase, an N-methyltransferase, an indole-ethylamine methyltransferase, a tryptamine 5-hydroxylase, and a dimethylallyl tryptamine synthase.
10. The microorganism of embodiment 9, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
11. The microorganism of embodiment 9, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
12. The microorganism of embodiment 9, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
13. The microorganism of embodiment 9, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
14. The microorganism of embodiment 9, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
15. The microorganism of embodiment 9, wherein the tryptamine 5-hydroxylase comprises an amino acid sequence with at least 80%, at least 85%, at least 90%, at least 95%, at least 99%, or 100% sequence identity to the sequence as shown in SEQ ID NO:
16. The microorganism of embodiment 9, wherein the dimethylallyl tryptamine synthase comprises an amino acid sequence with at least 80%, at least 85%, at least 90%, at least 95%, at least 99%, or 100% sequence identity to the sequence as shown in SEQ ID NO: 17.
17. A microorganism comprising at least one exogenous nucleic acid molecule that encodes at least one substituted tryptamine biosynthetic pathway enzyme.
18. The microorganism of embodiment 17, wherein the at least one substituted tryptamine biosynthetic pathway enzyme comprises a tryptophan decarboxylase, a tryptamine 4-monooxygenase, a 4-hydroxytryptamine kinase, and an N-methyltransferase.
19. The microorganism of embodiment 18, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
20. The microorganism of embodiment 17, wherein the at least one substituted tryptamine biosynthetic pathway enzyme comprises a tryptamine 4-monooxygenase, a 4-hydroxytryptamine kinase, and an N-methyltransferase.
21. The microorganism of embodiment 20, wherein the at least one substituted tryptamine biosynthetic pathway enzyme does not comprise a tryptophan decarboxylase.
22. The microorganism of embodiment 20 or 21, wherein the microorganism does not comprise an exogenous nucleic acid molecule that encodes a tryptophan decarboxylase.
23. The microorganism of any one of embodiments 18 to 22, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
24. The microorganism of any one of embodiments 18 to 23, wherein the 4-hydroxytryptamine kinase comprises an amino acid sequence with at least 80%, at leas85%, at least 90%, at least 95%, at least 99%, or 100% sequence identity to the sequence as shown in any of SEQ ID NOs: 8-12 and 43-59.
25. The microorganism of any one of embodiments 18 to 24, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
26. The microorganism of any one of embodiments 18 to 25, wherein the microorganism comprises at least one exogenous nucleic acid molecule that encodes a tryptamine 5-hydroxylase.
27. The microorganism of embodiment 26, wherein the tryptamine 5-hydroxylase comprises an amino acid sequence with at least 80%, at least 85%, at least 90%, at least 95%, at least 99%, or 100% sequence identity to the sequence as shown in SEQ ID NO:
28. The microorganism of any one of embodiments 18 to 27, wherein the microorganism comprises at least one exogenous nucleic acid molecule that encodes an indole-ethylamine methyltransferase.
29. The microorganism of embodiment 28, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
30. The microorganism of embodiment 17, wherein the at least one substituted tryptamine biosynthetic pathway enzyme comprises a tryptophan decarboxylase and a tryptamine 5-hydroxylase.
31. The microorganism of embodiment 30, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
32. The microorganism of embodiment 17, wherein the at least one substituted tryptamine biosynthetic pathway enzyme comprises a tryptamine 5-hydroxylase.
33. The microorganism of embodiment 32, wherein the at least one substituted tryptamine biosynthetic pathway enzyme does not comprise a tryptophan decarboxylase.
34. The microorganism of embodiment 32 or 33, wherein the microorganism does not comprise an exogenous nucleic acid molecule that encodes a tryptophan decarboxylase.
35. The microorganism of any one of embodiments 30 to 34, wherein the tryptamine 5-hydroxylase comprises an amino acid sequence with at least 80%, at least 85%, at least 90%, at least 95%, at least 99%, or 100% sequence identity to the sequence as shown in SEQ ID NO: 15.
36. The microorganism of embodiment 17, wherein the at least one substituted tryptamine biosynthetic pathway enzyme comprises a tryptophan decarboxylase and an indole-ethylamine methyltransferase.
37. The microorganism of embodiment 36, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
38. The microorganism of embodiment 17, wherein the at least one substituted tryptamine biosynthetic pathway enzyme comprises an indole-ethylamine methyltransferase.
39. The microorganism of embodiment 38, wherein the at least one substituted tryptamine biosynthetic pathway enzyme does not comprise a tryptophan decarboxylase.
40. The microorganism of embodiment 38 or 39, wherein the microorganism does not comprise an exogenous nucleic acid molecule that encodes a tryptophan decarboxylase.
41. The microorganism of any one of embodiments 36 to 40, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
42. A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, an indole-ethylamine methyltransferase, a tryptamine 4-monooxygenase, and a 4-hydroxytryptamine kinase.
43. The microorganism of embodiment 42, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
44. A microorganism comprising at least one exogenous nucleic acid molecule that encodes at least one substituted tryptamine biosynthetic pathway enzyme, wherein the at least one substituted tryptamine biosynthetic pathway enzyme comprises an indole-ethylamine methyltransferase, a tryptamine 4-monooxygenase, and a 4-hydroxytryptamine kinase.
45. The microorganism of embodiment 44, wherein the at least one substituted tryptamine biosynthetic pathway enzyme does not comprise a tryptophan decarboxylase.
46. The microorganism of embodiment 44 or 45, wherein the microorganism does not comprise an exogenous nucleic acid molecule that encodes a tryptophan decarboxylase.
47. The microorganism of any one of embodiments 42 to 46, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
48. The microorganism of any one of embodiments 42 to 47, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
49. The microorganism of any one of embodiments 42 to 48, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
50. A microorganism comprising at least one exogenous nucleic acid molecule that encodes at least one substituted tryptamine biosynthetic pathway enzyme, wherein the at least one substituted tryptamine biosynthetic pathway enzyme comprises a dimethylallyl tryptamine synthase.
51. The microorganism of embodiment 50, wherein the dimethylallyl tryptamine synthase comprises an amino acid sequence with at least 80%, at least 85%, at least 90%, at least 95%, at least 99%, or 100% sequence identity to the sequence as shown in SEQ ID NO: 17.
52. The microorganism of embodiment 50 or 51, wherein the at least one substituted tryptamine biosynthetic pathway enzyme further comprises an aurantioclavine synthase.
53. The microorganism of embodiment 52, wherein the aurantioclavine synthase comprises an amino acid sequence with at least 80%, at least 85%, at least 90%, at least 95%, at least 99%, or 100% sequence identity to the sequence as shown in SEQ ID NO: 18.
54. A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, a tryptamine 5-hydroxylase, and an indole-ethylamine methyltransferase.

The microorganism of embodiment 54, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
56. A microorganism comprising at least one exogenous nucleic acid molecule that encodes at least one substituted tryptamine biosynthetic pathway enzyme, wherein the at least one substituted tryptamine biosynthetic pathway enzyme comprises a tryptamine 5-hydroxylase, and an indole-ethylamine methyltransferase.
57. The microorganism of embodiment 56, wherein the at least one substituted tryptamine biosynthetic pathway enzyme does not comprise a tryptophan decarboxylase.
58. The microorganism of embodiment 56 or 57, wherein the microorganism does not comprise an exogenous nucleic acid molecule that encodes a tryptophan decarboxylase.
59. The microorganism of any one of embodiments 54 to 58, wherein the tryptamine 5-hydroxylase comprises an amino acid sequence with at least 80%, at least 85%, at least 90%, at least 95%, at least 99%, or 100% sequence identity to the sequence as shown in SEQ ID NO: 15.
60. The microorganism of any one of embodiments 54 to 59, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
61. A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.
62. The microorganism of embodiment 61, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
63. A microorganism comprising at least one exogenous nucleic acid molecule that encodes at least one substituted tryptamine biosynthetic pathway enzyme, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.
64. The microorganism of embodiment 63, wherein the at least one substituted tryptamine biosynthetic pathway enzyme does not comprise a tryptophan decarboxylase
65. The microorganism of embodiment 63 or 64, wherein the microorganism does not comprise an exogenous nucleic acid molecule that encodes a tryptophan decarboxylase.
66. The microorganism of any one of embodiments 61 to 65, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
67. The microorganism of any one of embodiments 61 to 66, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
68. The microorganism of any one of embodiments 61 to 67, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
69. The microorganism of any one of embodiments 61 to 67, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
70. A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan halogenase and a tryptophan decarboxylase.
71. The microorganism of embodiment 70, wherein the tryptophan halogenase comprises an amino acid sequence with at least 80%, at least 85%, at least 90%, at least 95%, at least 99%, or 100% sequence identity to the sequence as shown in any of SEQ ID NOs: 99-106.
72. The microorganism of embodiment 70 or 71, wherein the tryptophan decarboxylase comprises an amino acid sequence with at least 80%, at least 85%, at least 90%, at least 95%, at least 99%, or 100% sequence identity to the sequence as shown in any of SEQ ID NOs: 1-3 and 23-35.
73. The microorganism of any one of embodiments 71 to 72, wherein the at least one substituted biosynthetic pathway enzyme further comprises one or more substituted tryptamine biosynthetic pathway enzymes with at least 80%, at least 85%, at least 90%, at least 95%, at least 99%, or 100% sequence identity to the sequence as shown in any of SEQ ID NOs: 1-18, 23-98, and 107-321.
74. The microorganism of any one of embodiments 8 to 69, which is capable of producing at least one substituted tryptamine.
75. The microorganism of embodiment 70, wherein the at least one substituted tryptamine comprises one or more of serotonin, N-acetyl serotonin, dimethylallyl tryptamine, lysergic acid diethylamide, N-methyltryptamine, N,N-Dimethyltryptamine, N,N,N-Trimethyltryptamine, N,N,N-Trimethyl-4-phosphoryloxytryptamine (aeruginascin), psilocybin, psilocin, baeocystin, norbaeocystin, 4-hydroxytryptamine, N-acetyl-4-hydroxytrptamine, gramine, clavine, indole-acetic acid , ateviridine, Pindolol, bufotenin, aurantioclavine, and/or a halogenated substituted tryptamine.
76. The microorganism of any one of embodiments 17 to 75, wherein the at least one exogenous nucleic acid molecule is comprised in one or more episomal vectors.
77. The microorganism of any one of embodiments 17 to 76, further comprising increased expression of tryptophan synthase.
78. The microorganism of embodiment 77, wherein the expression of an endogenous tryptophan synthase is increased by altering culture conditions.
79. The microorganism of embodiment 78, wherein the altered culture conditions are nutrient limitation (e.g. phosphate deprivation, nitrogen deprivation, iron deprivation) and/or light deprivation (e.g. withdrawal of light sources, switching of light source spectra).
80. The microorganism of any one of embodiments 77 to 79, comprising an exogenous nucleic acid molecule that encodes the endogenous tryptophan synthase.
81. The microorganism of embodiment 80, wherein the endogenous tryptophan synthase encoded by the exogenous nucleic acid molecule comprises an amino acid sequence with at least 80%, at least 85%, at least 90%, at least 95%, at least 99%, or 100% sequence identity to the wild-type endogenous tryptophan synthase.
82. The microorganism of any one of embodiments 77 to 79, comprising an exogenous nucleic acid molecule that encodes an exogenous tryptophan synthase.
83. The microorganism of embodiment 82, wherein the exogenous tryptophan synthase comprises an amino acid sequence with at least 80%, at least 85%, at least 90%, at least 95%, at least 99%, or 100% sequence identity to the sequence as shown in any of SEQ ID NOs: 19-22.
84. The microorganism of any one of embodiments 1 to 83, wherein the microorganism is a microalga, a stramenopile, or a cyanobacteria.
85. The microorganism of embodiment 84, wherein the microalga is a Chlorophyceae, Trebouxiophyxeae, Coscinodiscophyceae, Bacillariphyceae, Eustigmatophyceae, or Labyrinthylomycetes.
86. The microorganism of embodiment 84, wherein the microalga is a Chlamydomonales, Chlorellales, Thalassiosirales, Baccilariales, Eustigmatales, or Labyrinthulales.
87. The microorganism of embodiment 84, wherein the microalga is a Chlamydomonas, Chlorella, Tetraselmis, Nannochloropsis, Phaeodactylum, Thalassiosira, Prototheca, Scenedesmus, Acutodesmus, Schizochytrium, Dunaliella, Aurantiochytrium, Thraustochytrium, Ulkenia, or Haematococus.
88. The microorganism of embodiment 84, wherein the microalgae is Chlamydomonas reinhardtii, Chlorella vulgaris, Chlorella sorokiniana, Chlorella protothecoides, Tetraselmis chuff, Nannochloropsis oculata, Phaeodactylum tricornutum, Thalassiosira pseudonana, Prototheca moriformis, Scenedesmus obliquus, Acutodesmus dimorphus, Schizochytrium limacinum, Dunaliella tertiolecta, Aurantiochytrium sp., Thraustochytrium sp., Ulkenia sp., or Heamatococus plucialis.
89. The microorganism of embodiment 84, wherein the microalga is Chlamydomonas reinhardtii.
90. The microorganism of embodiment 84, wherein the microalga is Phaeodactylum tricomutum.
91. The microorganism of embodiment 84, wherein the microalga is Schizochytrium limacinum.
92. The microorganism of embodiment 84, wherein the stramenopile is a Coscinodiscophyceae, Bacillariphyceae, Eustigmatophyceae, or Labyrinthylomycetes.
93. The microorganism of embodiment 84, wherein the stramenopile is a Thalassiosirales, Baccilariales, Eustigmatales, or Labyrinthulales.
94. The microorganism of embodiment 84, wherein the stramenopile is a Thalassiosira, Phaeodactylum, Nannochloropsis, Schizochytrium Aurantiochytrium, Thraustochytrium, or Ulkenia.
95. The microorganism of embodiment 84, wherein the stramenopile is Nannochloropsis oculata, Phaeodactylum tricomutum, Thalassiosira pseudonana, Schizochytrium limacinum, Aurantiochytrium sp., Thraustochytrium sp., or Ulkenia sp.
96. The microorganism of embodiment 84, wherein the stramenopile is Phaeodactylum tricomutum.
97. The microorganism of embodiment 84, wherein the stramenopile is Schizochytrium limacinum.
98. The microorganism of embodiment 84, wherein the microalgae is a diatom.
99. A substituted tryptamine biosynthetic pathway enzyme comprising an amino acid sequence with at least 80%, at least 85%, at least 90%, at least 95%, at least 99%, or 100% identity to the sequence as shown in any of SEQ ID NOs: 1-18 and 23-321.
100. A tryptophan synthase comprising an amino acid sequence with at least 80%, at least 85%, at least 90%, at least 95%, at least 99%, or 100% identity to the sequence as shown in any of SEQ ID NOs: 19-22.
101. A nucleic acid molecule encoding the enzyme of embodiment 99 or 100.
102. A vector comprising the nucleic acid molecule of embodiment 101.
103. A cell comprising the nucleic acid molecule of embodiment 101 or the vector of embodiment 102.
104. A cell culture comprising (i) the microorganism of any one of embodiments 17 to 98 and (ii) a culture media optionally supplemented with a high concentration of tryptamine.
105. A cell culture comprising (i) a microalga or a stramenopile comprising at least one exogenous nucleic acid molecule that encodes at least one substituted tryptamine biosynthetic pathway enzyme and (ii) a culture media supplemented with a high concentration of tryptamine.
106. The cell culture of embodiment 105, wherein the microalga is a Chlorophyceae, Trebouxiophyxeae, Coscinodiscophyceae, Bacillariphyceae, Eustigmatophyceae, or Labyrinthylomycetes.
107. The cell culture of embodiment 105, wherein the microalga is a Chlamydomonales, Chlorellales, Thalassiosirales, Baccilariales, Eustigmatales, or Labyrinthulales.
108. The cell culture of embodiment 105, wherein the microalga is a Chlamydomonas, Chlorella, Tetraselmis, Nannochloropsis, Phaeodactylum, Thalassiosira, Prototheca, Scenedesmus, Acutodesmus, Schizochytrium, Dunaliella, Aurantiochytrium, Thraustochytrium, Ulkenia, or Haematococus.
109. The cell culture of embodiment 105, wherein the microalga is Chlamydomonas reinhardtii, Chlorella vulgaris, Chlorella sorokiniana, Chlorella protothecoides, Tetraselmis chuff, Nannochloropsis oculata, Phaeodactylum tricornutum, Thalassiosira pseudonana, Prototheca moriformis, Scenedesmus obliquus, Acutodesmus dimorphus, Schizochytrium limacinum, Dunaliella tertiolecta, Aurantiochytrium sp., Thraustochytrium sp., Ulkenia sp., or Heamatococus plucialis.
110. The cell culture of embodiment 105, wherein the microalga is Chlamydomonas reinhardtii.
111. The cell culture of embodiment 105, wherein the microalga is Phaeodactylum tricornutum.
112. The cell culture of embodiment 105, wherein the microalga is Schizochytrium limacinum.
113. The cell culture of embodiment 105, wherein the stramenopile is a Coscinodiscophyceae, Bacillariphyceae, Eustigmatophyceae, or Labyrinthylomycetes.
114. The cell culture of embodiment 105, wherein the stramenopile is a Thalassiosirales, Baccilariales, Eustigmatales, or Labyrinthulales.
115. The cell culture of embodiment 105, wherein the stramenopile is a Thalassiosira, Phaeodactylum, Nannochloropsis, Schizochytrium Aurantiochytrium, Thraustochytrium, or Ulkenia.
116. The cell culture of embodiment 105, wherein the stramenopile is Nannochloropsis oculata, Phaeodactylum tricornutum, Thalassiosira pseudonana, Schizochytrium limacinum, Aurantiochytrium sp., Thraustochytrium sp., or Ulkenia sp.
117. The cell culture of embodiment 105, wherein the stramenopile is Phaeodactylum tricornutum.
118. The cell culture of embodiment 105, wherein the stramenopile is Schizochytrium limacinum.
119. The cell culture of any one of embodiments 104 to 118, wherein the high concentration of tryptamine is at least about 1 mM, 2 mM, 3 mM, 4 mM, 5 mM, 6 mM, 7mM, 9 mM, or 10 mM.
120. The cell culture of any one of embodiments 104 to 119, wherein the at least one substituted tryptamine biosynthetic pathway enzyme comprises a tryptamine 4-monooxygenase, a 4-hydroxytryptamine kinase, and an N-methyltransferase.
121. The cell culture of embodiment 120, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
122. The cell culture of embodiment 120 or 121, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
123. The cell culture of any one of embodiments 120 to 122, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
124. The cell culture of any one of embodiments 104 to 119, wherein the at least one substituted tryptamine biosynthetic pathway enzyme comprises a tryptamine 5-hydroxylase.
125. The cell culture of embodiment 124, wherein the tryptamine 5-hydroxylase comprises an amino acid sequence with at least 80%, at least 85%, at least 90%, at least 95%, at least 99%, or 100% sequence identity to the sequence as shown in SEQ ID NO:
126. The cell culture of any one of embodiments 104 to 119, wherein the at least one substituted tryptamine biosynthetic pathway enzyme comprises an indole-ethylamine methyltransferase.
127. The cell culture of embodiment 126, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
128. The cell culture of any one of embodiments 104 to 119, wherein the at least one substituted tryptamine biosynthetic pathway enzyme comprises an indole-ethylamine methyltransferase, a tryptamine 4-monooxygenase, and a 4-hydroxytryptamine kinase.
129. The cell culture of embodiment 128, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
130. The cell culture of embodiment 128 or 129, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
131. The cell culture of any one of embodiments 128 to 130, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
132. The cell culture of any one of embodiments 104 to 119, wherein the at least one substituted tryptamine biosynthetic pathway enzyme comprises a dimethylallyl tryptamine synthase and an aurantioclavine synthase.
133. The cell culture of embodiment 132, wherein the dimethylallyl tryptamine synthase comprises an amino acid sequence with at least 80%, at least 85%, at least 90%, at least 95%, at least 99%, or 100% sequence identity to the sequence as shown in SEQ ID NO: 17.
134. The cell culture of embodiment 132 or 133, wherein the aurantioclavine synthase comprises an amino acid sequence with at least 80%, at least 85%, at least 90%, at least 95%, at least 99%, or 100% sequence identity to the sequence as shown in SEQ ID NO: 18.
135. The cell culture of any one of embodiments 104 to 119, wherein the at least one substituted tryptamine biosynthetic pathway enzyme comprises a tryptamine 5-hydroxylase, and an indole-ethylamine methyltransferase.
136. The cell culture of embodiment 135, wherein the tryptamine 5-hydroxylase comprises an amino acid sequence with at least 80%, at least 85%, at least 90%, at least 95%, at least 99%, or 100% sequence identity to the sequence as shown in SEQ ID NO:
137. The cell culture of embodiment 135 or 136, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
138. The cell culture of any one of embodiments 104 to 119, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.
139. The cell culture of embodiment 138, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
140. The cell culture of embodiment 138 or 139 wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
141. The cell culture of any one of embodiments 138 to 140, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
142. The cell culture of any one of embodiments 138 to 140, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
143. The cell culture of any one of embodiments 105 to 142, wherein the at least one substituted tryptamine biosynthetic pathway enzyme does not comprise a tryptophan decarboxylase.
144. The cell culture of any one of embodiments 105 to 143, wherein the microorganism does not comprise an exogenous nucleic acid molecule that encodes a tryptophan decarboxylase.
145. The cell culture of any one of embodiments 105 to 144, wherein the microalga or a stramenopile produces at least one substituted tryptamine.
146. The cell culture of embodiment 145, wherein the at least one substituted tryptamine comprises one or more of serotonin, N-acetyl serotonin, dimethylallyl tryptamine, lysergic acid diethylamide, N-methyltryptamine, N,N-Dimethyltryptamine, N,N,N-Trimethyltryptamine, N,N,N-Trimethyl-4-phosphoryloxytryptamine (aeruginascin), psilocybin, psilocin, baeocystin, norbaeocystin, 4-hydroxytryptamine, N-acetyl-4-hydroxytrptamine, gramine, clavine, indole-acetic acid , ateviridine, Pindolol, bufotenin, and/or aurantioclavine.
147. The cell culture of any one of embodiments 105 to 146, wherein the cell culture undergoes autotrophic growth.
148. The cell culture of embodiment 147, wherein the autotrophic growth is photosynthetic growth.
149. The cell culture of embodiment 148, wherein the photosynthetic growth occurs in the presence of a solar light source.
150. The cell culture of embodiment 148, wherein the photosynthetic growth occurs in the presence of an artificial light source.
151. The cell culture of any one of embodiments 105 to 150, wherein the cell culture undergoes growth in organic conditions.
152. A method for producing at least one substituted tryptamine in a microalga or a stramenopile, comprising culturing the microalga or stramenopile in a culture media supplemented with a high concentration of tryptamine, wherein the microalga or stramenopile comprises at least one exogenous nucleic acid molecule that encodes at least one substituted tryptamine biosynthetic pathway enzyme.
153. The method of embodiment 152, wherein the microalga is a Chlorophyceae, Trebouxiophyxeae, Coscinodiscophyceae, Bacillariphyceae, Eustigmatophyceae, or Labyrinthylomycetes.
154. The method embodiment 152, wherein the microalga is a Chlamydomonales, Chlorellales, Thalassiosirales, Baccilariales, Eustigmatales, or Labyrinthulales.
155. The method of embodiment 152, wherein the microalga is a Chlamydomonas, Chlorella, Tetraselmis, Nannochloropsis, Phaeodactylum, Thalassiosira, Prototheca, Scenedesmus, Acutodesmus, Schizochytrium, Dunaliella, Aurantiochytrium, Thraustochytrium, Ulkenia, or Haematococus.
156. The method of embodiment 152, wherein the microalga is Chlamydomonas reinhardtii, Chlorella vulgaris, Chlorella sorokiniana, Chlorella protothecoides, Tetraselmis chuff, Nannochloropsis oculata, Phaeodactylum tricornutum, Thalassiosira pseudonana, Prototheca moriformis, Scenedesmus obliquus, Acutodesmus dimorphus, Schizochytrium limacinum, Dunaliella tertiolecta, Aurantiochytrium sp., Thraustochytrium sp., Ulkenia sp., or Heamatococus plucialis.
157. The method of embodiment 152, wherein the microalga is Chlamydomonas reinhardtii.
158. The method of embodiment 152, wherein the microalga is Phaeodactylum tricomutum.
159. The method of embodiment 152, wherein the microalga is Schizochytrium limacinum.
160. The method of embodiment 152, wherein the stramenopile is a Coscinodiscophyceae, Bacillariphyceae, Eustigmatophyceae, or Labyrinthylomycetes.
161. The method of embodiment 152, wherein the stramenopile is a Thalassiosirales, Baccilariales, Eustigmatales, or Labyrinthulales.
162. The method of embodiment 152, wherein the stramenopile is a Thalassiosira, Phaeodactylum, Nannochloropsis, Schizochytrium Aurantiochytrium, Thraustochytrium, or Ulkenia.
163. The method of embodiment 152, wherein the stramenopile is Nannochloropsis oculata, Phaeodactylum tricomutum, Thalassiosira pseudonana, Schizochytrium limacinum, Aurantiochytrium sp., Thraustochytrium sp., or Ulkenia sp.
164. The method of embodiment 152, wherein the stramenopile is Phaeodactylum tricomutum.
165. The method of embodiment 152, wherein the stramenopile is Schizochytrium limacinum.
166. The method of any one of embodiments 152 to 165, wherein the high concentration of tryptamine is at least about 1 mM, 2 mM, 3 mM, 4 mM, 5 mM, 6 mM, 7 mM, 9 mM, or 10 mM.
167. The method of any one of embodiments 152 to 166, wherein the at least one substituted tryptamine biosynthetic pathway enzyme comprises a tryptamine 4-monooxygenase, a 4-hydroxytryptamine kinase, and an N-methyltransferase.
168. The method of embodiment 167, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
169. The method of embodiment 167 or 168, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
170. The method of any one of embodiments 167 to 169, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
171. The method of any one of embodiments 152 to 166, wherein the at least one substituted tryptamine biosynthetic pathway enzyme comprises a tryptamine 5-hydroxylase.
172. The method of embodiment 171, wherein the tryptamine 5-hydroxylase comprises an amino acid sequence with at least 80%, at least 85%, at least 90%, at least 95%, at least 99%, or 100% sequence identity to the sequence as shown in SEQ ID NO: 15.
173. The method of any one of embodiments 152 to 166, wherein the at least one substituted tryptamine biosynthetic pathway enzyme comprises an indole-ethylamine methyltransferase.
174. The method of embodiment 173, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
175. The method of any one of embodiments 152 to 166, wherein the at least one substituted tryptamine biosynthetic pathway enzyme comprises an indole-ethylamine methyltransferase, a tryptamine 4-monooxygenase, and a 4-hydroxytryptamine kinase.
176. The method of embodiment 175, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
177. The method of embodiment 175 or 176, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
178. The method of any one of embodiments 175 to 177, wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
179. The method of any one of embodiments 152 to 166, wherein the at least one substituted tryptamine biosynthetic pathway enzyme comprises a dimethylallyl tryptamine synthase and an aurantioclavine synthase.
180. The method of embodiment 179, wherein the dimethylallyl tryptamine synthase comprises an amino acid sequence with at least 80%, at least 85%, at least 90%, at least 95%, at least 99%, or 100% sequence identity to the sequence as shown in SEQ ID NO: 17.
181. The method of embodiment 179 or 180, wherein the aurantioclavine synthase comprises an amino acid sequence with at least 80%, at least 85%, at least 90%, at least 95%, at least 99%, or 100% sequence identity to the sequence as shown in SEQ ID NO: 18.
182. The method of any one of embodiments 152 to 166, wherein the at least one substituted tryptamine biosynthetic pathway enzyme comprises a tryptamine 5-hydroxylase, and an indole-ethylamine methyltransferase.
183. The method of embodiment 182, wherein the tryptamine 5-hydroxylase comprises an amino acid sequence with at least 80%, at least 85%, at least 90%, at least 95%, at least 99%, or 100% sequence identity to the sequence as shown in SEQ ID NO: 15.
184. The method of embodiment 182 or 183, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
185. The method of any one of embodiments 152 to 166, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.
186. The method of embodiment 185, wherein the tryptamine 4-monooxygenase comprises an amino acid sequence with at least 80%, at least 85%, at least 90%, at least 95%, at least 99%, or 100% sequence identity to the sequence as shown in any of SEQ ID NOs: 4-7 and 36-42.
187. The method of embodiment 185 or 186 wherein the 4-hydroxytryptamine kinase comprises an amino acid sequence with at least 80%, at least 85%, at least 90%, at least 95%, at least 99%, or 100% sequence identity to the sequence as shown in any of SEQ ID NOs: 8-12 and 43-59.
188. The method of any one of embodiments 186 to 187, wherein the N-methyltransferase comprises an amino acid sequence with at least 80%, at least 85%, at least 90%, at least 95%, at least 99%, or 100% sequence identity to the sequence as shown in any of SEQ ID NOs: 13-14 and 109-321.
189. The method of any one of embodiments 186 to 188, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.
190. The method of any one of embodiments 152 to 189, wherein the at least one substituted tryptamine biosynthetic pathway enzyme does not comprise a tryptophan decarboxylase.
191. The method of any one of embodiments 152 to 190, wherein the microorganism does not comprise an exogenous nucleic acid molecule that encodes a tryptophan decarboxylase.
192. The method of any one of embodiments 152 to 191, wherein the at least one substituted tryptamine comprises one or more of serotonin, N-acetyl serotonin, dimethylallyl tryptamine, lysergic acid diethylamide, N-methyltryptamine, N,N-Dimethyltryptamine, N,N,N-Trimethyltryptamine, N,N,N-Trimethyl-4-phosphoryloxytryptamine (aeruginascin), psilocybin, psilocin, baeocystin, norbaeocystin, 4-hydroxytryptamine, N-acetyl-4-hydroxytrptamine, gramine, clavine, indole-acetic acid , ateviridine, Pindolol, bufotenin, and/or aurantioclavine.
193. The method of any one of embodiments 152 to 192, wherein the microalga or stramenopile undergoes autotrophic growth.
194. The method of embodiment 193, wherein the autotrophic growth is photosynthetic growth.

195. The method of embodiment 194, wherein the photosynthetic growth occurs in the presence of a solar light source.

196. The method of embodiment 194, wherein the photosynthetic growth occurs in the presence of an artificial light source.

197. The method of any one of embodiments 152 to 196, further comprising isolating the at least one substituted tryptamine.

### EXAMPLE 1

Microorganisms were cultured separately in varying concentrations of supplemented tryptamine to test tolerance and growth rate. Log-phase cultures of Phaeodactylum tricornutum Schizochytrium limacinum, Chlamydomonas reihardtii, Saccharomyces cerevisiae, Escherichia coli were each supplemented with 0 mM, 0.1 mM, 0.5 mM, 1 mM, 2 mM, 5 mM, or 10 mM of tryptamine.

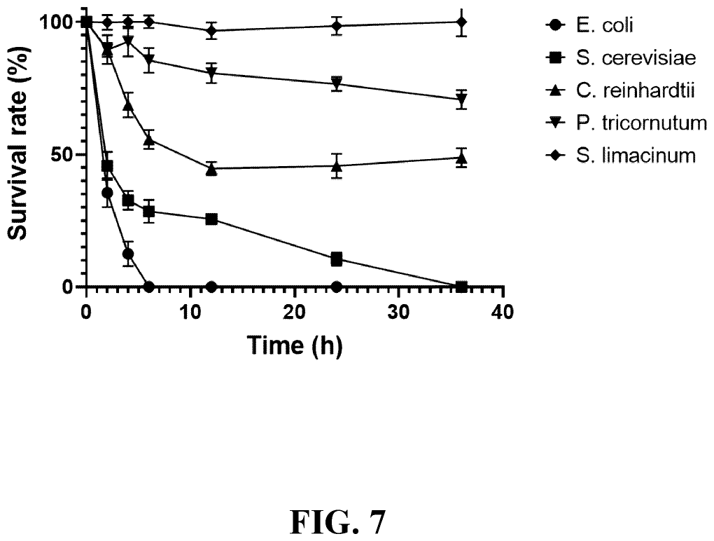

FIG. 7 shows the survival rate of each microorganism over 36 hours following addition of tryptamine at a concentration of 2mM to the culture media, as measured by trypan-blue exclusion. Briefly, 50 μL of cell culture was incubated for 5 min with equal volume of 0.4% trypan blue (SigmaAldrich). At least three independent experiments were performed with more than 500 cells counted per condition. For validation, live-dead assay was also conducted using fluorescent dyes FDA/PI in Biotek Synergy H1 plate reader.

The cultures of E. coli and S. cerevisiae were dead within 4 hours and 36 hours following supplementation, respectively. By contrast, the cultures of C. reinhardtii, P. tricornutum and S. limacinum tolerated the tryptamine supplementation and maintained viabilities of ˜45%, ˜75% and ˜99%, respectively.

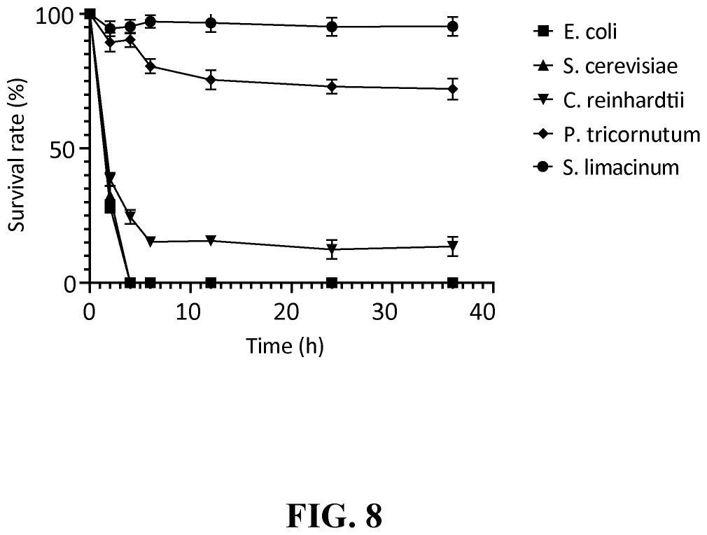

FIG. 8 shows the survival rate of each microorganism over 36 hours following addition of tryptamine at a concentration of 5 mM to the culture media, as measured by trypan-blue exclusion. The cultures of E. coli and S. cerevisiae were dead within 4 hours following supplementation. By contrast, the cultures of C. reinhardtii, P. tricornutum and S. limacinum tolerated the tryptamine supplementation and maintained viabilities of ˜15%, ˜75% and ˜95%, respectively.

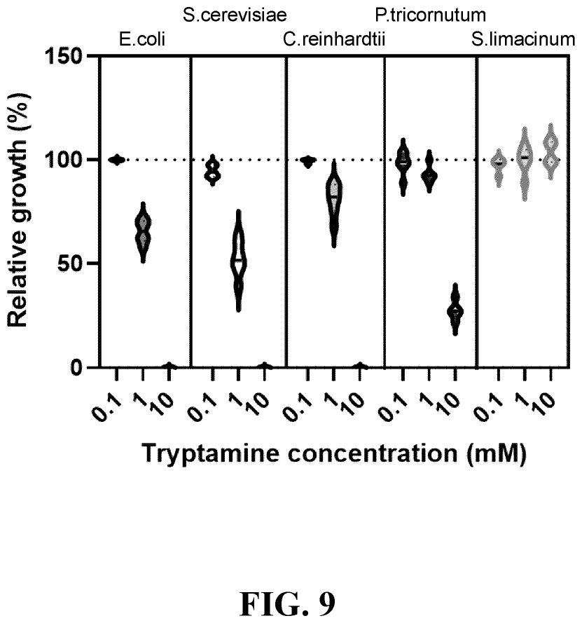

FIG. 9 shows the relative growth of each microorganism by 16 hours (E. coli and S. cerevisiae) or 36 hours (C. reinhardtii, P. tricornutum, and S. limacinum) after the addition of tryptamine at 0.1 mM, 1 mM, or 10 mM to the culture media. Growth of E. coli and S. cerevisiae were significantly diminished at a tryptamine concentration of 1 mM, whereas C. reinhardtii maintained ˜75% of normal growth. The growth rate of P. tricornutum was not affected until 5 mM and the growth rate of S. limacinum was not affected at any tested concentration.

These results show that microalgae (e.g. C. reinhardtii and P. tricornutum) and stramenopiles (e.g. P. tricornutum and S. limacinum) tolerate high concentrations of tryptamine that kill or severely restrict growth of yeast and bacteria, and are therefore useful for high-yield biosynthetic production of tryptamine derivatives in tryptamine-supplemented cultures.

1. A microalga or stramenopile comprising at least one exogenous nucleic acid molecule that encodes at least one substituted tryptamine biosynthetic pathway enzyme, wherein the microalga or stramenopile does not comprise an exogenous nucleic acid molecule encoding tryptophan decarboxylase.

2. The microalga or stramenopile of claim 1, wherein the at least one substituted tryptamine biosynthetic pathway enzyme comprises a tryptamine 4-monooxygenase, a 4-hydroxytryptamine kinase, and an N-methyltransferase.

3. The microalga or stramenopile of claim 1, wherein the at least one substituted tryptamine biosynthetic pathway enzyme comprises a tryptamine 5-hydroxylase.

4. The microalga or stramenopile of claim 1, wherein the at least one substituted tryptamine biosynthetic pathway enzyme comprises an indole-ethylamine methyltransferase.

5. The microalga or stramenopile of claim 1, wherein the at least one substituted tryptamine biosynthetic pathway enzyme comprises an indole-ethylamine methyltransferase, a tryptamine 4-monooxygenase, and a 4-hydroxytryptamine kinase.

6. The microalga or stramenopile of claim 1, wherein the at least one substituted tryptamine biosynthetic pathway enzyme comprises a dimethylallyl tryptamine synthase.

7. The microalga or stramenopile of claim 1, wherein the at least one substituted tryptamine biosynthetic pathway enzyme comprises a tryptamine 5-hydroxylase and an indole-ethylamine methyltransferase.

8. The microalga or stramenopile of claim 1, wherein the at least one substituted tryptamine biosynthetic pathway enzyme comprises a tryptamine 4-monooxygenase, a 4-hydroxytryptamine kinase, and either an N-methyltransferase or an indole-ethylamine methyltransferase, wherein the N-methyltransferase or indole-ethylamine methyltransferase is overexpressed and/or present in multiple copies.

9. The microalga or stramenopile of claim 1, which is Chlamydomonas reinhardtii, Phaeodactylum tricornutum, or Schizochytrium limacinum.

10. The microalga or stramenopile of claim 1, which is capable of producing at least one substituted tryptamine.

11. A microorganism comprising at least one exogenous nucleic acid molecule that encodes at least one substituted tryptamine biosynthetic pathway enzyme, wherein the at least one substituted tryptamine biosynthetic pathway enzyme comprises a tryptophan decarboxylase, an indole-ethylamine methyltransferase, a tryptamine 4-monooxygenase, and a 4-hydroxytryptamine kinase.

12. The microorganism of claim 11, wherein the indole-ethylamine methyltransferase comprises an amino acid sequence with at least 80%, at least 85%, at least 90%, at least 95%, at least 99%, or 100% sequence identity to the sequence as shown in any of SEQ ID NOs: 16 and 60-98.

13. The microorganism of claim 11, which is a microalga.

14. The microorganism of claim 11, which is capable of producing at least one substituted tryptamine.

15. A cell culture comprising (i) a microalga or stramenopile comprising at least one exogenous nucleic acid molecule that encodes at least one substituted tryptamine biosynthetic pathway enzyme and (ii) a culture media supplemented with a high concentration of tryptamine.

16. The cell culture of claim 15, wherein the microalga or stramenopile is Chlamydomonas reinhardtii, Phaeodactylum tricornutum, or Schizochytrium limacinum.

17. The cell culture of claim 15, wherein the high concentration of tryptamine is at least about 1 mM, 2 mM, 3 mM, 4 mM, 5 mM, 6 mM, 7 mM, 9 mM, or 10 mM.

18. The cell culture of claim 15, wherein the at least one substituted tryptamine biosynthetic pathway enzyme comprises a tryptamine 4-monooxygenase, a 4-hydroxytryptamine kinase, and an N-methyltransferase.

19. The microorganism of claim 15, wherein the microorganism or stramenopile does not comprise an exogenous nucleic acid molecule that encodes a tryptophan decarboxylase.

20. The cell culture of claim 15, wherein the microalga or stramenopile produces at least one substituted tryptamine.
